(19) United States
(12) Patent Application Publication
(10) Pub. No.: US 2017/0049401 A1
(43) Pub. Date: Feb. 23, 2017

(54) WEARABLE PHYSIOLOGICAL MEASUREMENT DEVICE AND SIGNAL COMPARISON METHOD THEREOF

(71) Applicant: Cheng Uei Precision Industry Co., Ltd., New Taipei City (TW)

(72) Inventor: James Cheng Lee, New Taipei City (TW)

(21) Appl. No.: 15/343,260

(22) Filed: Nov. 4, 2016

Related U.S. Application Data
(62) Division of application No. 14/448,758, filed on Jul. 31, 2014.

(51) Int. Cl. A61B 5/00; A61B 5/1455; A61B 5/024

(52) U.S. Cl. CPC A61B 5/7214; A61B 5/6824; A61B 5/02416; A61B 5/02438; A61B 5/6831; A61B 5/742; A61B 5/14551; A61B 5/02405

(57) ABSTRACT

A wearable physiological measurement device worn on an arm of a body includes an electronic module, at least one strap body electrically connected with the electronic module, a first sensing module electrically connected with the processor module, and a second sensing module electrically connected with the processor module. An inside of the electronic module is equipped with a processor module. An inner surface of the strap body is flush with an inner surface of the electronic module. The first sensing module is fastened to the electronic module and is exposed to the inner surface of the electronic module to be close to a skin surface of an outer side of the arm. The second sensing module is fastened to the strap body and is exposed to an inner surface of the strap body to be closed to a skin surface of an inner side of the arm.

### CROSS-REFERENCES TO RELATED APPLICATIONS

This is a division of U.S. patent application Ser. No. 14/448,758, filed Jul. 31, 2014, which is incorporated herewith by reference in its entirety.

### BACKGROUND OF THE INVENTION

1. Field of the Invention

The present invention generally relates to a physiological measurement device, and more particularly to a wearable physiological measurement device and a signal comparison method thereof

2. The Related Art

Generally, a current wearable physiological measurement device is used for recording a variation of photoplethysmography for determining heart rates. The function of the wearable physiological measurement device is realized by virtue of at least one light source and an optical sensor. The variation of photoplethysmography is generated by the optical sensor capturing a reflected light source which is reflected by body tissues after the light source irradiates the body tissues. The variation of photoplethysmography is an intensity change of the reflected light source to measure the heart rates.

The measurement method of the wearable physiological measurement device has characteristics of non-invasive performance, real-time performance and high accuracy, so the wearable physiological measurement device is liked by many sporters. In the exercise process, a wearable physiological measurement device is used to be able to record pulse changes throughout the whole process, disclose intense degrees of sporters regulating their own bodies, or serve as references of exercise plans.

But the optical sensor of the current wearable physiological measurement device is disposed on a skin surface of an outer side of an arm of a user, so in some intense exercises, such as running or rope skipping, the wearable physiological measurement device will sustain continuous and violent shaking to make the optical sensor capture ambient light sources, and cause acoustic noise signals of the ambient light sources captured by the optical sensor of the wearable physiological measurement device. In the process of running or rope skipping, the wearable physiological measurement device will be in continuous shaking statues to cause the variation of photoplethysmography captured by the wearable physiological measurement device to sustain continuous interferences of the acoustic noise signals.

Generally, a fastening structure or a shading structure which is fitted to body is designed as far as possible for preventing the wearable physiological measurement device from leaking light. But skin is a soft tissue, the skin will be deformed under the violent shaking, usually, the shading structure will still leak light under the burning sun, and a contact area of the skin which the wearable physiological measurement device contacts is increased by the shading structure to cause a comfort level of wearing the wearable physiological measurement device to be lowered. So under the dynamic condition, it is still unable to block the generated acoustic noise signals completely, and the above problem has become the most happened problem of the wearable physiological measurement device.

However, colors of the skin surfaces of the outer sides of the arms of most users appear to be darker, especially colored people are more obvious. If the colors of the skin surfaces of the outer sides of the arms of the users are darker or more glossy, after the light sources irradiate the skin surface of the outer side of the arm, conditions of the light source hardly penetrating through the skin surface of the outer side of the arm or the light source being reflected without the light source penetrating through the skin surface of the outer side of the arm are caused, the variation of photoplethysmography which is able to be captured is weaker that makes physiological information measured by the wearable physiological measurement device generate an error. Thus, the utility of the wearable physiological measurement device will be affected.

In order to improve measurement accuracies of the wearable physiological measurement device in most conditions, herein an innovative wearable physiological measurement device is provided for the present invention, and the innovative wearable physiological measurement device is capable of conquering the errors generated by leaking light and the color of the skin surface of the arm in the measurement process for improving the utility of the wearable physiological measurement device.

### SUMMARY OF THE INVENTION

An object of the present invention is to provide a wearable physiological measurement device and a signal comparison method thereof The wearable physiological measurement device worn on an arm of a body, includes an electronic module, at least one strap body, a first sensing module and a second sensing module. An inside of the electronic module is equipped with a processor module. The strap body is electrically connected with the electronic module for fastening the electronic module to the arm of the body. An inner surface of the strap body is flush with an inner surface of the electronic module at the time of the electronic module and the strap body being disposed horizontally. The first sensing module is fastened to the electronic module and is exposed to the inner surface of the electronic module to be close to a skin surface of an outer side of the arm. The first sensing module electrically connected with the processor module, includes at least one first light source, and at least one first optical sensor. The second sensing module is fastened to the strap body and is exposed to an inner surface of the strap body to be closed to a skin surface of an inner side of the arm. The second sensing module electrically connected with the processor module, includes at least one second light source and at least one second optical sensor.

The signal comparison method of a wearable physiological measurement device includes specific steps described hereinafter. A first optical sensor and a second optical sensor of the wearable physiological measurement device capture and transmit signals to a processor module of the wearable physiological measurement device. The processor module compares a first signal-to-noise ratio of the signal with a second signal-to-noise ratio of the signal, when the first signal-to-noise ratio is larger than the second signal-to-noise ratio, the processor module turns off a power of a second sensing module of the wearable physiological measurement device and calculates a first variation of photoplethysmography, or when the first signal-to-noise ratio is smaller than the second signal-to-noise ratio, the processor module turns off a power of a first sensing module of the wearable physiological measurement device, and calculates a second variation of photoplethysmography until a specific sensing time is over to terminate sensing.

The signal comparison method of a wearable physiological measurement device includes another specific steps described hereinafter. A first optical sensor and a second optical sensor of the wearable physiological measurement device capture and transmit signals to a processor module of the wearable physiological measurement device. The processor module compares a first signal-to-noise ratio of the signal with a signal-to-noise limit of the signal, when the first signal-to-noise ratio is larger than the signal-to-noise limit, the processor module turns off a power of a second sensing module of the wearable physiological measurement device, and calculates a first variation of photoplethysmography until a specific sensing time is over, or when the first signal-to-noise ratio is smaller than the signal-to-noise limit, the processor module compares the first signal-to-noise ratio of the signal with a second signal-to-noise ratio of the signal, calculates a variation of photoplethysmography with the larger signal-to-noise ratio, and simultaneously, the processor module turns off the sensing module which is a first sensing module of the wearable physiological measurement device or the second sensing module with the smaller signal-to-noise ratio, and calculates a variation of photoplethysmography which is a second variation of photoplethysmography or the first variation of photoplethysmography until the specific sensing time is over to terminate sensing.

As described above, the wearable physiological measurement device is capable of solving the common problem thereof to make dynamic measurement values corresponding to static measurement values, so the wearable physiological measurement device is fit for being used under exercises. Furthermore, a color of the skin surface of the inner side of the arm is usually lighter than a color of the skin surface of the outer side of the arm, after the light source irradiates the skin surface of the inner side of the arm, conditions of the light source hardly penetrating through the skin surface of the inner side of the arm or the light source being reflected without the light source penetrating through the skin surface of the inner side of the arm are caused, the variation of photoplethysmography which is able to be captured is normal. Thus, the wearable physiological measurement device is capable of proceeding measuring through the second sensing module which is close to the skin surface of the inner side of the arm by virtue of the signal comparison method thereof for improving the utility of the wearable physiological measurement device.

### BRIEF DESCRIPTION OF THE DRAWINGS

The present invention will be apparent to those skilled in the art by reading the following description, with reference to the attached drawings, in which:

FIG. 1 is a perspective view of a wearable physiological measurement device in accordance with an embodiment of the present invention;

FIG. 2 is a principle block diagram of the wearable physiological measurement device of FIG. 1;

FIG. 3 is a schematic diagram of the wearable physiological measurement device of FIG. 1, wherein the wearable physiological measurement device is worn on an arm;

FIG. 4 is a flowchart of a signal comparison method of the wearable physiological measurement device of FIG. 1; and

FIG. 5 is a flowchart of another signal comparison method of the wearable physiological measurement device of FIG. 1.

### DETAILED DESCRIPTION OF THE PREFERRED EMBODIMENT

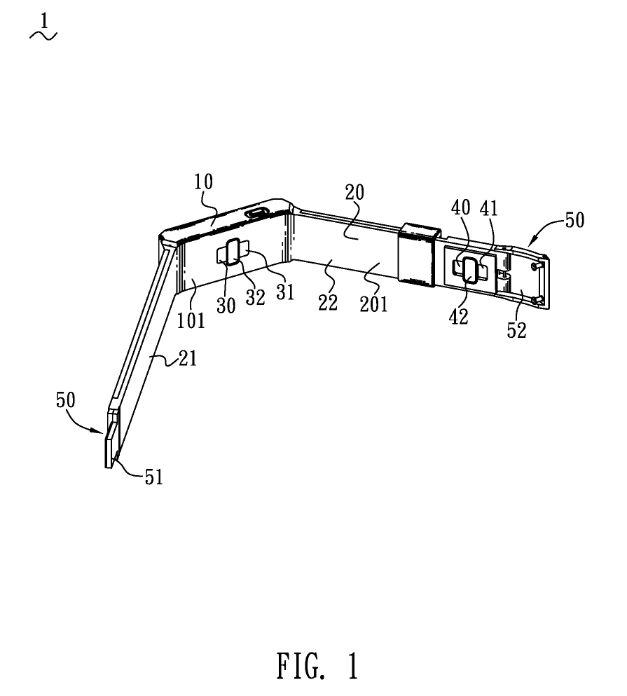

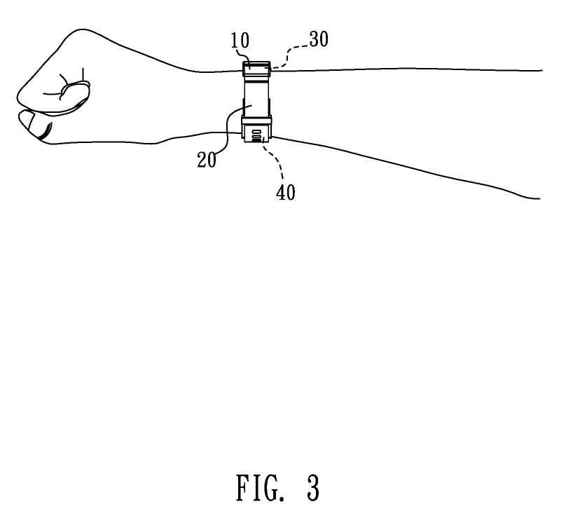

Referring to FIG. 1 and FIG. 3, a wearable physiological measurement device 1 in accordance with an embodiment of the present invention is shown. The wearable physiological measurement device 1 worn on an arm of a body, includes an electronic module 10, at least one strap body 20, a first sensing module 30 and a second sensing module 40. The strap body 20 is electrically connected with the electronic module 10 for fastening the electronic module 10 to the arm of the body. The first sensing module 30 is fastened to the electronic module 10. The second sensing module 40 is fastened to the strap body 20. The first sensing module 30 and the second sensing module 40 are used for capturing physiological information.

Referring to FIG. 1 and FIG. 3 again, an inner surface 201 of the strap body 20 is flush with an inner surface 101 of the electronic module 10 at the time of the electronic module 10 and the strap body 20 being disposed horizontally. The strap body 20 has at least one part made of insulating material for fastening the electronic module 10 to the arm of the body. In the embodiment, the strap body 20 includes a first strap body 21 and a second strap body 22 oppositely fastened to two ends of the electronic module 10 for fastening the electronic module 10 to the arm of the body. The strap body 20 has buckling structures 50 which include a first buckling structure 51 disposed at a tail end of the first strap body 21, and a second buckling structure 52 disposed at a tail end of the second strap body 22. The tail ends of the first strap body 21 and the second strap body 22 are buckled with each other by virtue of the first buckling structure 51 being buckled with the second buckling structure 52. In this embodiment, the buckling structures 50 are cramp ring structures, thread gluing structures or folding clasp structures.

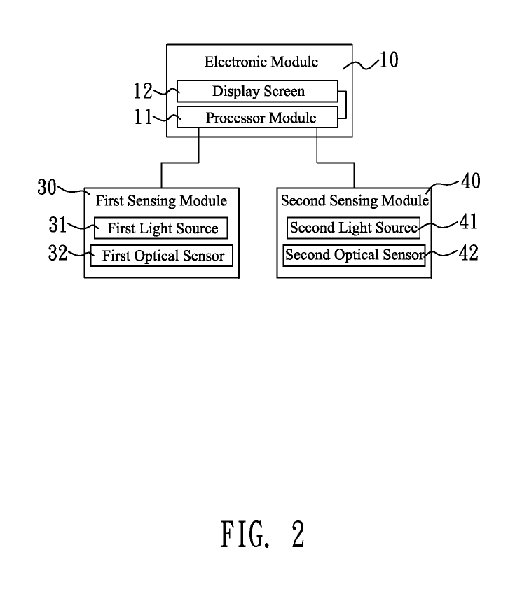

Referring to FIG. 2, an inside of the electronic module 10 is equipped with a processor module 11. An outer side of the electronic module 10 is equipped with a display screen 12. The processor module 11 is electrically connected with the first sensing module 30 and the second sensing module 40 for calculating the physiological information captured by the first sensing module 30 and the second sensing module 40. The display screen 12 is electrically connected with the processor module 11 for displaying the physiological information.

Referring to FIG. 1, FIG. 2 and FIG. 3, the first sensing module 30 is fastened to the electronic module 10 and is exposed to the inner surface 101 of the electronic module 10 to be closed to a skin surface of an outer side of the arm of the body of a user. The first sensing module 30 electrically connected with the processor module 11, includes at least one first light source 31, and at least one first optical sensor 32.

Referring to FIG. 1, FIG. 2 and FIG. 3, the second sensing module 40 is fastened to the strap body 20 and is exposed to the inner surface 201 of the strap body 20 to be closed to a skin surface of an inner side of the arm of the body of the user. The second sensing module 40 electrically connected with the processor module 11, includes at least one second light source 41 and at least one second optical sensor 42. In this embodiment, the first optical sensor 32 and the second optical sensor 42 are photodiodes or phototransistors.

Referring to FIG. 1 and FIG. 3, when the wearable physiological measurement device 1 is worn on the arm of the user, the first sensing module 30 and the second sensing module 40 are respectively located at the two opposite sides of the arm of the user. When the wearable physiological measurement device 1 is worn on the arm of the body of the user, the first sensing module 30 faces to the skin surface of the outer side of the arm, and the second sensing module 40 faces to the skin surface of the inner side of the arm.

Referring to FIG. 1, FIG. 2 and FIG. 3, when the wearable physiological measurement device 1 is worn on the arm of the body of the user, the first sensing module 30 is close to the skin surface of the outer side of the arm of the user so that the first light source 31 irradiates the skin surface of the outer side of the arm, and the first optical sensor 32 is capable of capturing a first reflected light source reflected by the skin surface of the outer side of the arm. The second sensing module 40 is close to the skin surface of the inner side of the arm of the user so that the second light source 41 irradiates the skin surface of the inner side of the arm, and the second optical sensor 42 is capable of capturing a second reflected light source reflected by the skin surface of the inner side of the arm. A first signal of the first reflected light source captured by the first optical sensor 32 and a second signal of the second reflected light source captured by the second optical sensor 42 are respectively transmitted to the processor module 11.

Referring to FIG. 2, after the first optical sensor 32 and the second optical sensor 42 input the signals which include variations of photoplethysmography, signal-to-noise ratios and performance indexes to the processor module 11, the processor module 11 can measure the variations of photoplethysmography, the signal-to-noise ratios and the performance indexes according to the signals. The signals include the first signals transmitted by the first optical sensor 32, and the second signals transmitted by the second optical sensor 42. The variation of photoplethysmography is a change of blood flow per unit area in a blood vessel. The variation of photoplethysmography records a condition of blood reflecting the light source.

Referring to FIG. 2 again, with differences of oxygen contents of the blood, absorbing degrees of the light sources are different. When a heart is contracted, the oxygen content of the blood will be increased, on the contrary, when the heart is expanded, the oxygen content of the blood will be decreased. So an intensity of the light source captured by the optical sensor is the variation of photoplethysmography. The light source is the first light source 31 or the second light source 41. The optical sensor is the first optical sensor 32 or the second optical sensor 42.

A signal-to-noise ratio is used for distinguishing numerical values of signal transmission qualities. The signal-to-noise ratio is a ratio between an average value of the signal and a standard deviation of the signal. The smaller the signal-to-noise ratio is, the larger the noise is, so a signal accuracy is worse, on the contrary, the larger the signal-to-noise ratio is, the smaller the noise is, so the signal accuracy is higher.

A performance index is a difference value between an alternating voltage level and a direct voltage level shown in a heartbeat waveform. The larger the performance index is, the more obvious the measured signal is, so the signal accuracy is higher, on the contrary, the smaller the performance index is, the less obvious the measured signal is, so the signal accuracy is worse.

Referring to FIG. 2, so the first signals transmitted by the first optical sensor 32 include a first variation of photoplethysmography, a first signal-to-noise ratio and a first performance index, and the second signals transmitted by the second optical sensor 42 include a second variation of photoplethysmography, a second signal-to-noise ratio and a second performance index. Correspondingly, the variations of photoplethysmography include the first variation of photoplethysmography and the second variation of photoplethysmography. The signal-to-noise ratios include the first signal-to-noise ratio and the second signal-to-noise ratio. The performance indexes include the first performance index and the second performance index.

Referring to FIG. 2, the processor module 11 is capable of distinguishing which is more accurate, the first variation of photoplethysmography or the second variation of photoplethysmography by virtue of comparing the first signal-to-noise ratio of the first optical sensor 32 with the second signal-to-noise ratio of the second optical sensor 42 or comparing the first performance index of the first optical sensor 32 with the second performance index of the second optical sensor 42. Thereby the processor module 11 chooses the better variation of photoplethysmography to calculate the physiological information which includes heart rates, the oxygen contents of blood, heart rate variabilities and so on.

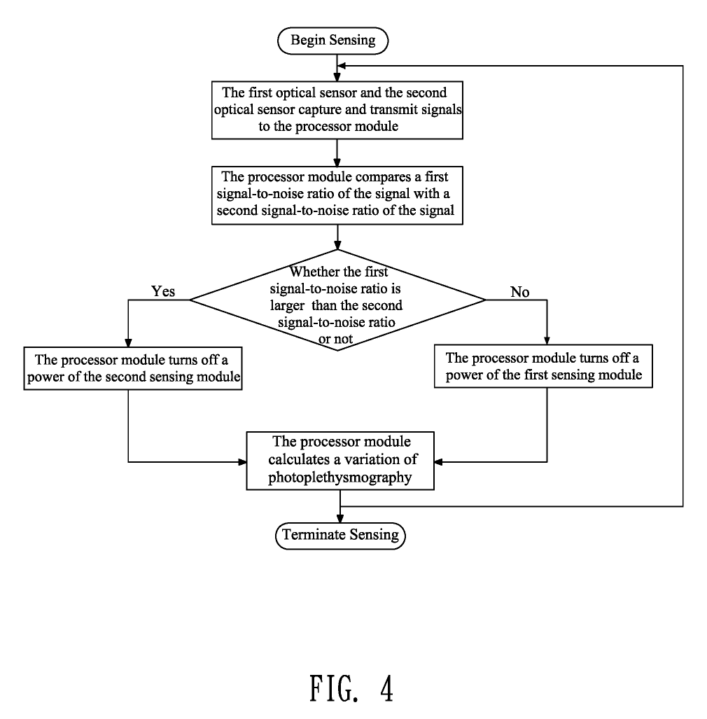

Referring to FIG. 1, FIG. 2 and FIG. 4, specific steps of a signal comparison method of the wearable physiological measurement device 1 are described as follows, and include a specific sensing time, the sensing time is within a few seconds or within a few milliseconds, after beginning sensing:

The first optical sensor 32 and the second optical sensor 42 capture and transmit the signals to the processor module 11.

The processor module 11 compares the first signal-to-noise ratio of the signal with the second signal-to-noise ratio of the signal, when the first signal-to-noise ratio is larger than the second signal-to-noise ratio, the processor module 11 turns off a power of the second sensing module 40 and calculates the first variation of photoplethysmography, or when the first signal-to-noise ratio is smaller than the second signal-to-noise ratio, the processor module 11 turns off a power of the first sensing module 30, and calculates the second variation of photoplethysmography until the specific sensing time is over to terminate sensing.

The steps of the above-mentioned signal comparison method are repeated to make the processor module 11 be able to calculate multiple groups of the continuous-time variations of photoplethysmography for getting the more accurate physiological information.

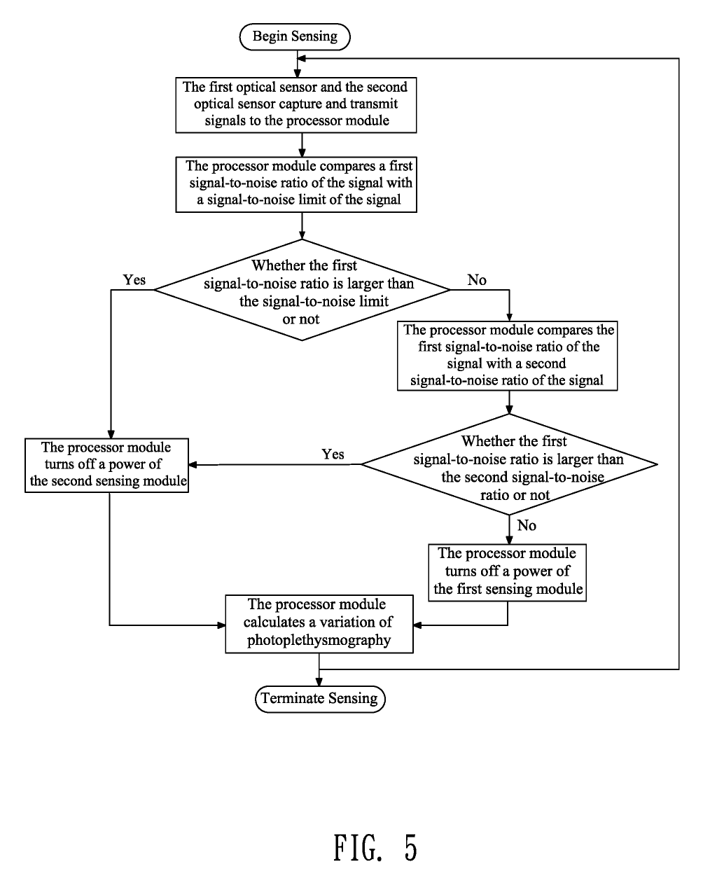

Referring to FIG. 1, FIG. 2 and FIG. 5, specific steps of another signal comparison method of the wearable physiological measurement device 1 are described as follows, and include the specific sensing time, the sensing time is within a few seconds or within a few milliseconds, after beginning sensing:

The first optical sensor 32 and the second optical sensor 42 capture and transmit the signals to the processor module 11.

The processor module 11 compares the first signal-to-noise ratio of the signal with a signal-to-noise limit of the signal, when the first signal-to-noise ratio is larger than the signal-to-noise limit, the processor module 11 turns off the power of the second sensing module 40, and calculates the first variation of photoplethysmography until the specific sensing time is over, or when the first signal-to-noise ratio is smaller than the signal-to-noise limit, the processor module 11 compares the first signal-to-noise ratio of the signal with the second signal-to-noise ratio of the signal, calculates the variation of photoplethysmography with the larger signal-to-noise ratio, and simultaneously, the processor module 11 turns off the sensing module which is the first sensing module 30 or the second sensing module 40 with the smaller signal-to-noise ratio, and calculates the variation of photoplethysmography which is the second variation of photoplethysmography or the first variation of photoplethysmography until the specific sensing time is over to terminate sensing.

The steps of the above-mentioned signal comparison method are repeated to make the processor module 11 be able to get the multistage continuous variations of photoplethysmography under the specific sensing time for getting the physiological information.

In the signal comparison method, besides comparing the signal-to-noise ratios, the processor module 11 is also able to compare the performance indexes or simultaneously compare the signal-to-noise ratios and the performance indexes for getting distinguishing basises.

The signal-to-noise ratio limit is the smallest signal-to-noise ratio value of being able to correctly judge a physiological data of the variation of photoplethysmography.

Referring to FIG. 1, FIG. 2 and FIG. 3, in the process of the wearable physiological measurement device 1 being worn by the user, regardless of running or intense exercises, the wearable physiological measurement device 1 is able to correctly calculates the variation of photoplethysmography of the proper sensing module by virtue of a structure thereof and the signal comparison method thereof Under the status of wearing the wearable physiological measurement device 1, at the time of running or intense exercises, soft tissues of the arm of the user are deformed or a clearance is formed between the sensing module which is the first sensing module 30 or the second sensing module 40 and the skin surface when the wearable physiological measurement device 1 is shaken by sustaining a centrifugal force, at the moment, the environmental signal-to-noise ratio signals will be captured by the sensing module, usually, in this situation, the wearable physiological measurement device 1 is able to make the processor module 11 choose the more accurate signal so that the physiological information is more accurate by virtue of the signal comparison method.

Referring to FIG. 1, FIG. 2 and FIG. 3, when a clearance is formed between the first sensing module 30 and the skin surface of the outer side of the arm, it will cause the second sensing module 40 to be close to the skin surface of the inner side of the arm. When a clearance is formed between the second sensing module 40 and the skin surface of the inner side of the arm, it will cause the first sensing module 30 to be close to the skin surface of the outer side of the arm. The phenomenon is because the worn strap body 20 has a constant length structure when the wearable physiological measurement device 1 is shaken. So if the clearance is formed between one sensing module facing to one side of the arm and the skin surface of the side of the arm, the other sensing module facing to the other side of the arm will be pulled to close to the skin surface of the other side of the arm by the strap body 20, so the pulled sensing module is closer to the skin surface of the other side of the arm.

Referring to FIG. 1, FIG. 2 and FIG. 3, in other word, when the arm is shaken reciprocally and intensely, the first sensing module 30 and the second sensing module 40 are respectively thrown in turn on account of the first sensing module 30 and the second sensing module 40 sustaining inertias of their own gravities. Usually, in the process of the first sensing module 30 and the second sensing module 40 being thrown, the sensing modules are pulled to close to the skin surfaces of the two opposite sides of the arm. So if the clearance is formed between the skin surface of one side of the arm and one sensing module facing to the skin surface of the side of the arm, the other sensing module is closer to the skin surface of the other side of the arm.

Referring to FIG. 1, FIG. 2 and FIG. 3, because the sensing module is closer to the skin surface, the smaller the signal-to-noise ratio is, the more accurate the measured physiological information is. So it can be known from the motions, the accuracy of the wearable physiological measurement device 1 will be without affection by the shaking under the intense exercises.

Referring to FIG. 1, FIG. 2 and FIG. 3, after the first sensing module 30 is thrown towards an outside, the clearance is formed between the first sensing module 30 and the skin surface of the outer side of the arm, so the signal-to-noise ratios and the performance indexes got by the first sensing module 30 will be decreased, so the second sensing module 40 will be close to the skin surface of the inner side of the arm, the signal-to-noise ratios and the performance indexes will be increased, so the processor module 11 calculates the second variation of photoplethysmography of the second sensing module 40 by virtue of the signal comparison method. On the contrary, the clearance between the second sensing module 40 and the skin surface of the inner side of the arm, the processor 11 calculates the first variation of photoplethysmography of the first sensing module 30 by virtue of the signal comparison method, thereby the accuracy of the physiological information is improved sharply.

As described above, the wearable physiological measurement device 1 is capable of solving the common problem thereof to make dynamic measurement values corresponding to static measurement values, so the wearable physiological measurement device 1 is fit for being used under exercises. Furthermore, a color of the skin surface of the inner side of the arm is usually lighter than a color of the skin surface of the outer side of the arm, after the light source irradiates the skin surface of the inner side of the arm, conditions of the light source hardly penetrating through the skin surface of the inner side of the arm or the light source being reflected without the light source penetrating through the skin surface of the inner side of the arm are caused, the variation of photoplethysmography which is able to be captured is normal. Thus, the wearable physiological measurement device 1 is capable of proceeding measuring through the second sensing module 40 which is close to the skin surface of the inner side of the arm by virtue of the signal comparison method thereof for improving the utility of the wearable physiological measurement device 1.

The forgoing description of the present invention has been presented for purposes of illustration and description. It is not intended to be exhaustive or to limit the invention to the precise form disclosed, and obviously many modifications and variations are possible in light of the above teaching. Such modifications and variations that may be apparent to those skilled in the art are intended to be included within the scope of this invention as defined by the accompanying claims.

What is claimed is:

1. A signal comparison method of a wearable physiological measurement device, comprising the steps of:
a first optical sensor and a second optical sensor of the wearable physiological measurement device capturing and transmitting signals to a processor module of the wearable physiological measurement device; and
the processor module comparing a first signal-to-noise ratio of the signal with a second signal-to-noise ratio of the signal, when the first signal-to-noise ratio is larger than the second signal-to-noise ratio, the processor module turning off a power of a second sensing module of the wearable physiological measurement device and calculating a first variation of photoplethysmography, or when the first signal-to-noise ratio is smaller than the second signal-to-noise ratio, the processor module turning off a power of a first sensing module of the wearable physiological measurement device, and calculating a second variation of photoplethysmography until a specific sensing time is over to terminate sensing.

2. The signal comparison method as claimed in claim 1, wherein the steps of the signal comparison method are repeated to make the processor module be able to get the multistage continuous variations of photoplethysmography under the specific sensing time for getting physiological information.

3. The signal comparison method as claimed in claim 1, wherein the specific sensing time is within a few seconds or within a few milliseconds.

4. The signal comparison method as claimed in claim 1, wherein the signals include variations of photoplethysmography, signal-to-noise ratios and performance indexes, the variations of photoplethysmography include the first variation of photoplethysmography and the second variation of photoplethysmography, the signal-to-noise ratios include the first signal-to-noise ratio and the second signal-to-noise ratio, the performance indexes include a first performance index and a second performance index.

5. The signal comparison method as claimed in claim 4, wherein the signals include first signals transmitted by the first optical sensor, and second signals transmitted by the second optical sensor, the first signals include the first variation of photoplethysmography, the first signal-to-noise ratio and the first performance index, and the second signals include the second variation of photoplethysmography, the second signal-to-noise ratio and the second performance index.

6. The signal comparison method as claimed in claim 4, wherein besides comparing the signal-to-noise ratios, the processor module is also able to compare the performance indexes or simultaneously compare the signal-to-noise ratios and the performance indexes for getting distinguishing basises.

7. A signal comparison method of a wearable physiological measurement device, comprising the steps of:
a first optical sensor and a second optical sensor of the wearable physiological measurement device capturing and transmitting signals to a processor module of the wearable physiological measurement device; and
the processor module comparing a first signal-to-noise ratio of the signal with a signal-to-noise limit of the signal, when the first signal-to-noise ratio is larger than the signal-to-noise limit, the processor module turning off a power of a second sensing module of the wearable physiological measurement device, and calculating a first variation of photoplethysmography until a specific sensing time is over, or when the first signal-to-noise ratio is smaller than the signal-to-noise limit, the processor module comparing the first signal-to-noise ratio with a second signal-to-noise ratio of the signal, calculating a variation of photoplethysmography with the larger signal-to-noise ratio, and simultaneously, the processor module turning off the sensing module which is a first sensing module of the wearable physiological measurement device or the second sensing module with the smaller signal-to-noise ratio, and calculating a variation of photoplethysmography which is a second variation of photoplethysmography or the first variation of photoplethysmography until the specific sensing time is over to terminate sensing.

8. The signal comparison method as claimed in claim 7, wherein the steps of the signal comparison method are repeated to make the processor module be able to get the multistage continuous variations of photoplethysmography under the specific sensing time for getting physiological information.

9. The signal comparison method as claimed in claim 7, wherein the specific sensing time is within a few seconds or within a few milliseconds.

10. The signal comparison method as claimed in claim 7, wherein the signal-to-noise ratio limit is the smallest signal-to-noise ratio value of being able to correctly judge a physiological data of the variation of photoplethysmography.

11. The signal comparison method as claimed in claim 7, wherein the signals include variations of photoplethysmography, signal-to-noise ratios and performance indexes, the variations of photoplethysmography include a first variation of photoplethysmography and a second variation of photoplethysmography, the signal-to-noise ratios include a first signal-to-noise ratio and a second signal-to-noise ratio, the performance indexes include a first performance index and a second performance index.

12. The signal comparison method as claimed in claim 11, wherein the signals include first signals transmitted by the first optical sensor, and second signals transmitted by the second optical sensor, the first signals include the first variation of photoplethysmography, the first signal-to-noise ratio and the first performance index, and the second signals include the second variation of photoplethysmography, the second signal-to-noise ratio and the second performance index.

13. The signal comparison method as claimed in claim 11, wherein besides comparing the signal-to-noise ratios, the processor module is also able to compare the performance indexes or simultaneously compare the signal-to-noise ratios and the performance indexes for getting distinguishing basises.
